(19) United States
(12) Patent Application Publication
(10) Pub. No.: US 2014/0163109 A1
(43) Pub. Date: Jun. 12, 2014

(54) TASTE-MASKED IBUPROPEN GRANULES

(71) Applicant: GLATT AG, Pratteln (CH)

(72) Inventors: Frédéric Gerber, Blotzheim (FR); Marie Guhmann, Huningue (FR)

(73) Assignee: Glatt Ag, Pratteln (CH)

(21) Appl. No.: 14/239,995

(22) PCT Filed: Mar. 26, 2013
(86) PCT No.: PCT/EP2013/000908

§ 371 Date: Feb. 20, 2014

(30) Foreign Application Priority Data
Mar. 26, 2012 (EP) 12002105.0

(51) Int. Cl. A61K 9/16; A61K 31/192

(52) U.S. Cl. CPC A61K 9/1617; A61K 31/192; A61K 9/1682
USPC 514/570; 427/2.14

(57) ABSTRACT

Taste-masked Ibuprofen granules and a process for preparation thereof, as well as an oral dosage form including such taste-masked Ibuprofen granules and the use of said granules in an oral dosage form.

The present application claims priority from PCT Patent Application No. PCT/EP2013/000908 filed on Mar. 26, 2013, which claims priority from European Patent Application No. EP 12002105.0 filed on Mar. 26, 2012, the disclosures of which are incorporated herein by reference in their entirety.

### FIELD OF THE INVENTION

The present invention is directed to taste-masked Ibuprofen granules and to a process for preparation thereof. Still further, the present invention is also directed to an oral dosage form comprising said granules and to the use of said granules in an oral dosage form.

It is noted that citation or identification of any document in this application is not an admission that such document is available as prior art to the present invention.

Ibuprofen, namely (±)-2-(4-isobutylphenyl) propionic acid, is a well-known drug with analgesic, anti-inflammatory and and-pyretic properties. Ibuprofen is available primarily for the treatment of painful and anti-inflammatory disorders including rheumatoid arthritis, ankylosing spondylitis, osteoarthritis, postoperative pain, post-partum pain and soft tissue injuries, generally administered to humans at doses of up to 3200 mg per day. Ibuprofen is a white powder or crystal, which is poorly soluble in water.

One disadvantage of Ibuprofen is its bad taste. Particularly, there is a bitter taste and an unbearable burning in the throat which appears at the moment of taking it and remains for an extended period.

Accordingly, there is a need of masking the taste of Ibuprofen in oral dosage forms. In the prior art there are several different examples for oral dosage forms, some of which containing taste-masked Ibuprofen. Typical dosage forms are those, with a taste-masking outer layer, e.g. tablets or capsules.

US 2003/0170312 A1 discloses a method for coating solid particles with a thermofusible agent. The solid particles are fluidized to obtain a homogeneous individualized distribution of the particles in an air fluidized bed. The thermofusible agent is melted and sprayed on the particles in the form of atomized droplets. The resulting coated particles are cooled so as to solidify the thermofusible agent around the particles. A preferred particle to be coated is an Ibuprofen particle, which is coated with fatty acid esters.

US 2003/0232097 A1 discloses an oily wax matrix suspension pharmaceutical formulation for oral administration of an active ingredient, such as Ibuprofen, through a soft gelatin capsule drug delivery device. The active pharmaceutical ingredient, preferably Ibuprofen as free acid and/or alkali salt form, is embedded in an oily wax matrix which is preferably blended with a surfactant. A preferred surfactant is lecithin, a preferred suspending agent is yellow beeswax, and a preferred suspension medium is soybean oil.

U.S. Pat. No. 5,180,590 discloses a particular effervescent aqueous solution of Ibuprofen obtained from effervescent granules and tablets made by dry granulation of 200 mg of Ibuprofen, 2100 mg of sodium bicarbonate and 500 mg of citric acid in a fluidized bed. The solution obtained after total decomposition of the tablets has a tolerable taste. Particularly, it is not bitter and does not cause irritation of the throat.

WO 00/24385 A1 discloses a taste-masked chewable tablet with instant release of the active principle characterized in that it consists of a mixture comprising individualized active principle particles coated with a lipid agent obtained by a reaction between at least a polyhydroxy alcohol and at least one free or esterified saturated fatty acid, said fatty acid comprising from 8 to 22 carbon atoms. Said mixture further comprises at least one disintegrating binder.

WO 2011/095814 A1 discloses orally administrable pharmaceutical compositions for treating respiratory disorders. Ibuprofen is formulated in combination with a lipophilic pharmaceutically acceptable vehicle comprising a lipid, preferably at least 30 wt %, and an alcohol. Upon oral administration, bioavailability of Ibuprofen in the lung of mice is increased.

JP 56-120616 A discloses melt granulates containing at least 78 wt % of Ibuprofen and pharmaceutical excipients. After cooling and solidification of the melt, the granulate is ground to give Ibuprofen-containing granules. Microcrystalline cellulose and calcium stearate are used as excipients in the Examples, the latter in an amount of less than 1 wt %. It is the object to obtain dense Ibuprofen granules to reduce the size of the final product.

EP 362 728 A2 discloses a granular composition of Ibuprofen for direct tableting. Ibuprofen is molten and solidified on a contact cooling apparatus using a seeding process and then comminuted. The formed granulate consists of Ibuprofen. Excipients for tableting are added afterwards.

U.S. Pat. No. 5,240,712 discloses fused unit dose compositions of Ibuprofen, optionally containing pharmaceutically acceptable excipients, as a solid solution and/or dispersion in Ibuprofen. A preferred dosage form is a hard gelatin capsule.

U.S. Pat. No. 5,667,807 discloses a thermal granulation process for the production of directly tablettable granules. The process is characterized in that a mixture of active compound and the necessary auxiliaries is processed by means of a melt extrusion at elevated temperature to give a homogeneous non-agglutinating extrudate which is then comminuted to give tablettable granules. By means of the mixing and kneading elements of the extruder, the mixture is compacted to give an extrudate at a temperature at which a part of the active compound is melted. By means of this process, apart from the lubricant, all auxiliaries such as binder, disintegration auxiliaries, fillers and other auxiliaries can be incorporated directly into the granules. The lubricant is added afterwards. The examples demonstrate that the extrusion temperature is below the melting point of the active compound, e.g. Ibuprofen. Magnesium stearate is used as a lubricant.

WO 99/40943 A1 discloses that certain actives, such as Ibuprofen, will form an eutectic material with certain solubilizers when processed under sufficient forces (such as shear forces, centrifugal forces or pressure) and at temperatures from below the formation temperature of the eutectic to below the temperature at which the active dissolves in the solubilizer, or melts. These eutectics, while in the presence of the temperature and force, will coat or envelop, at least partially, particles of the active (solubilizing delivery system). This solubilizing delivery system contains a higher percentage of active than the eutectic alone is capable of while still retaining the enhanced dissolution properties of the eutectic or a combination product of the solubilizer and active.

WO 01/41733 A2 discloses that if a disintegrating agent is incorporated into a molten NSAID like Ibuprofen and intimately combined therewith and then is cooled and milled to produce a granule, a composition capable of tabletting with minimum tabletting excipients and having advantageous tabletting, disintegration and dissolution properties is provided, if silicon dioxide is incorporated therein. It is preferred that the granular component of melt granules is combined with an extra-granular component. The extra-granular component comprises the ingredients incorporated in the compressed tablet which are not contained in the solidified melt granules. Preferably, the silicon dioxide is present in the extra-granular component. The extra-granular component may comprise lubricants such as stearic acid in an amount of up to 5 wt %.

WO 02/098392 discloses similar subject-matter. Instead of silicon dioxide it discloses a wicking agent in general. Said wicking agent is part of the extra-granular component and may comprise stearic acid or an insoluble salt thereof in an amount of up to 6 wt %.

U.S. Pat. No. 6,210,710 B1 discloses a pharmaceutical composition which releases the medicament for a prolonged or sustained period of time and can be formulated into many dosage forms. It is a blend of at least first and second components and a medicament. The first component is selected from hydroxypropylcellulose (HPC), ethylcellulose (EC), or derivatives of HPC, EC, and hydroxyethylcellulose (HEC) and the second component is at least one other polymer. Ibuprofen is one of the examples in a list of suitable medicaments. Said document further discloses that the pharmaceutical composition may comprise one or more lubricating agents, e.g., stearic acid, colloidal silicon dioxide, magnesium stearate, calcium stearate, waxes, polyethylene glycol, or magnesium lauryl sulfate, present in an amount of from about 0.25 to about 3 wt % of the total weight of the coated dosage form.

US 2010/0152486 A1 discloses a particular method for production of particles from a melt. The particles are formed from pharmaceutical substances (medicinal substances and/or pharmaceutical excipients), e.g. Ibuprofen.

U.S. Pat. No. 5,405,617 discloses pharmaceutical compositions and oral dosage forms made thereof wherein the pharmaceutical active is coated or combined with a taste masking or matrix forming effective amount of an aliphatic of fatty acid ester wherein the ratio of drug to aliphatic of fatty acid ester is 5:95 to 50:50 and wherein the ratios with lesser drug load provide taste masking. Ibuprofen is one of the possible pharmaceutical actives.

WO 96/29057 discloses pharmaceutical matrix pellets providing an adequate drug release profile and comprising drug particles, a hydrophilic compound and a hydrophobic compound. Ibuprofen is one of the possible pharmaceutical actives.

WO 97/40821 discloses the preparation of oral dosage units by directly tableting so-called shearlite particles which are prepared by subjecting a solid organic-based feedstock to liquiflash and shearing conditions. Ibuprofen is one of the possible pharmaceutical actives.

U.S. Pat. No. 6,071,539 discloses effervescent granules and methods for their preparation. The granules consist of an acidic agent, an alkaline agent and a hot-melt extrudable binder. Also a pharmaceutical tablet comprising the effervescent granules and a therapeutic compound is disclosed. Ibuprofen is one of the possible therapeutic compounds.

Roblegg et al. discloses in European Journal of Pharmaceutics and Biopharmaceutics 2010, 75, 1, 56-62 the development of lipophilic calcium stearate pellets using Ibuprofen as model drug. The pellets are formed by wet extrusion.

EP 0 582 380 A1 discloses a process for the production of spherical particles or granules by dry granulation, e.g. including Ibuprofen. Taste-masking is achieved by providing an additional coating.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

It is further noted that the invention does not intend to encompass within the scope of the invention any previously disclosed product, process of making the product or method of using the product, which meets the written description and enablement requirements of the USPTO (35 U.S.C. 112, first paragraph) or the EPO (Article 83 of the EPC), such that applicant(s) reserve the right to disclaim, and hereby disclose a disclaimer of any previously described product, method of making the product, of process of using the product.

### SUMMARY OF THE INVENTION

The present invention is based on the surprising finding that taste-masked Ibuprofen granules ate obtainable by hot-melt granulation of Ibuprofen and at least one pharmaceutically acceptable excipient.

As far as reference is made herein to a melt granulate, this shall denote the granular product received from a hot-melt granulation process.

### BRIEF DESCRIPTION OF THE DRAWINGS

FIG. 1 shows a fluid bed apparatus usable in the context of the present invention.

FIG. 2 shows the particle size distribution of the granules of Example 1.

FIG. 3 shows the particle size distribution of the granules of Example 2.

FIG. 4 shows the particle size distribution of the granules of Example 3.

FIG. 5 shows the particle size distribution of the granules of Example 4.

FIG. 6 shows the particle size distribution of the granules of Example 5.

FIG. 7 shows the particle size distribution of the granules of Example 6.

FIG. 8 shows the particle size distribution of the granules of Example 7.

FIG. 9 shows the release profile of the granules of Example 3.

FIG. 10 shows the release profile of the granules of Example 4.

FIG. 11 shows the release profile of the granules of Example 5.

FIG. 12 shows the release profile of the granules of Example 6.

FIG. 13 shows the release profile of the granules of Example 7.

### DETAILED DESCRIPTION OF EMBODIMENTS

It is to be understood that the figures and descriptions of the present invention have been simplified to illustrate elements that are relevant for a clear understanding of the present invention, while eliminating, for purposes of clarity, many other elements which are conventional in this art. Those of ordinary skill in the art will recognize that other elements are desirable for implementing the present invention. However, because such elements are well known in the art, and because they do not facilitate a better understanding of the present invention, a discussion of such elements is not provided herein.

The present invention will now be described in detail on the basis of exemplary embodiments.

Accordingly, the present invention in one aspect is directed to a melt granulate comprising Ibuprofen and a pharmaceutically acceptable excipient,
wherein said pharmaceutically acceptable excipient is selected from the group consisting of fatty acids, fatty acid salts, fatty acid esters, glyceride esters, waxes, polyvinyl caprolactam-polyvinyl acetate polyethylene glycol graft copolymer, or a combination of two or more thereof,
wherein said pharmaceutically acceptable excipient is present in the melt granulate in an amount of at least 10 wt % based on the total weight of the melt granulate, and
wherein Ibuprofen is present in the melt granulate preferably in an amount of at least 60 wt % based on the total weight of the melt granulate.

The granules according to the present invention contain Ibuprofen in a taste-masked manner, i.e. the granules exhibit appropriate taste properties. Hence, the problem of bad taste of Ibuprofen is overcome by the present invention without the need of any additional taste-masking layer to be applied onto the surface of the granules or onto the surface of a tablet comprising the granules.

The granules according to the present invention show immediate release characteristics. This is a particular important property since the granules are at the same time taste-masked. Hence, by simply providing a melt granulate comprising Ibuprofen and a pharmaceutically acceptable excipient as defined above in an amount of at least 10 wt % based on the total weight of the melt granulate, granules are obtained which contain Ibuprofen in a taste-masked manner and which have immediate release characteristics.

Immediate release solid oral dosage forms provide the release of most, or all, of the active pharmaceutical ingredient over a short period of time, such as 60 minutes or less, and make rapid absorption of the drug possible.

The pharmaceutically acceptable excipient defined above may be present in the melt granulate according to the present invention in an amount of at least 12 wt %, or in an amount of at least 15 wt %, or in an amount of at least 20 wt %, based on the total weight of the melt granulate. However, it is usually desired that the amount of Ibuprofen is sufficiently high. Therefore, the pharmaceutically acceptable excipient is usually present in an amount of not more than 40 wt %, e.g. not more than 30 wt % or not more than 25 wt % based on the total weight of the melt granulate.

Preferably, Ibuprofen is present in the melt granulate according to the present invention in an amount of at least 65 wt %, more preferably in an amount of at least 70 wt %, even more preferably in an amount of at least 75 wt % based on the total weight of the melt granulate.

Ibuprofen may be present in the melt granulate according to the present invention in an amount of up to 80 wt % or higher, e.g. around 85 wt %, based on the total weight of the melt granulate. However, the amount of Ibuprofen will usually be not higher than 90 wt % based on the total weight of the melt granulate.

Preferably, the sum of the amount of Ibuprofen and the amount of said pharmaceutically acceptable excipient is at least 90 wt %, more preferably at least 95 wt %, even more preferably at least 97 wt %, and even more preferably at least 99.5 wt % based on the total weight of the melt granulate.

Hence, preferably, there is no need for of any further compound being present in the melt granulate according to the present invention apart from Ibuprofen and said pharmaceutically acceptable excipient. Said two compounds are sufficient for providing taste-masked Ibuprofen granules according to the present invention. Therefore, preferably, the melt granulate according to the present invention essentially consists of more preferably consists of, Ibuprofen and said pharmaceutically acceptable excipient. The latter definition allows the presence of impurities which are present in the reactants and/or are formed in the step of preparation of the melt granulate in an amount of up to 0.5 wt % for the sum of all impurities based on the total weight of the melt granulate.

Preferably, in the fatty acids, fatty acid salts and fatty acid esters, usable as pharmaceutically acceptable excipient according to the present invention, the fatty acid part is a saturated fatty acid. More preferably, the fatty acid part contains between 8 and 26 carbon atoms, still more preferably between 12 and 24 carbon atoms.

Preferably, the glyceride esters usable as pharmaceutically acceptable excipient according to the present invention are natural glyceride esters. More preferably, said natural glyceride esters consist of esters of glycerin and fatty acids, more preferably saturated fatty acids. Still more preferably, the fatty acids contain between 8 and 26 carbon atoms each, still more preferably between 12 and 24 carbon atoms each.

Preferably, the waxes usable as pharmaceutically acceptable excipient according to the present invention are plant waxes. More preferably, said plant waxes consist in an amount of at least 80 wt % based on the total weight of the wax of members of the group selected from the following four classes of compounds: aliphatic esters, diesters of 4-hydroxycinnamic acid, Ω-hydroxycarboxylic acids and fatty acid alcohols. More preferably, the alcohol and acid parts in the members of said four classes of compounds contain between 26 and 38 carbon atoms each, still more preferably between 26 and 32 carbon atoms each.

Hence, according to the present invention, the pharmaceutically acceptable excipient is preferably selected from the group consisting of fatty acids, fatty acid salts and fatty acid esters, wherein the fatty acid part is a saturated fatty acid containing between 8 and 26 carbon atoms, still more preferably between 12 and 24 carbon atoms; natural glyceride esters consisting of esters of glycerin and saturated fatty acids containing between 8 and 26 carbon atoms each, still more preferably between 12 and 24 carbon atoms each; plant waxes which consist in an amount of at least 80 wt % based on the total weight of the wax of members of the group selected from the following four classes of compounds: aliphatic esters, diesters of 4-hydroxycinnamic acid, Ω-hydroxycarboxylic acids and fatty acid alcohols, and the alcohol and acid parts in the members of said four classes of compounds contain between 26 and 38 carbon atoms each, still more preferably between 26 and 32 carbon atoms each; and polyvinyl caprolactam-polyvinyl acetate polyethylene glycol graft copolymer; or a combination of two or more thereof.

Suitable members of the preferred classes of pharmaceutically acceptable excipients according to the present invention are stearic acid, Carnauba wax, polyvinyl caprolactam-polyvinyl acetate polyethylene glycol graft copolymer, or a combination of two or more thereof.

Preferably, said pharmaceutically acceptable excipient has a melting point of between 40 and 150° C., more preferably of between 45 and 120° C., and still more preferably of between 50 and 100° C.

Ibuprofen is a hydrophobic compound. Also the pharmaceutically acceptable excipients according to the present invention are hydrophobic or in case of polyvinyl caprolactam-polyvinyl acetate polyethylene glycol graft copolymer have amphiphilic properties. There is no need that the melt granulate according to the present invention comprises a considerable amount of any hydrophilic compound. Accordingly, the melt granulate according to the present invention preferably comprises less than 10 wt %, more preferably less than 5 wt %, and even more preferably less than 1 wt % based on the total weight of the melt granulate of any hydrophilic compound. It is even more preferred that the melt granulate according to the present invention does not comprise any hydrophilic compound. Consequently, the melt granulate according to the present invention preferably comprises at least 90 wt %, more preferably at least 95 wt %, and even more preferably at least 99 wt % based on the total weight of the melt granulate of hydrophobic, or hydrophobic and amphiphilic compounds. It is even more preferred that the melt granulate according to the present invention essentially consists of, more preferably consists of, hydrophobic, or hydrophobic and amphiphilic compounds.

Preferably, more than 90 wt %, more preferably more than 95 wt %, still more preferably more than 97 wt %, and particularly preferred more than 99 wt % of the melt granulate according to the present invention consists of granules having a length-width ratio of less than 1.4, more preferably less than 1.3, and still more preferably less than 1.2. Hence, it is particularly preferred that more than 99 wt % of the melt granulate according to the present invention consists of granules having a length-width ratio of less than 1.2.

Preferably, in the melt granulate according to present invention the average particle size of the granules is in the range from 0.05 to 1.0 mm. It is also preferred that more than 99 wt % of the melt granulate according to the present invention consists of granules having a particle size in the range from 0.05 to 1.0 mm. The particle size distribution is determined by sieve analysis.

The melt granulate according to the present invention may be produced by any hot-melt granulation process. Such processes are known to the person skilled in the art.

However, it is preferred that the melt granulate according to the present invention is produced by a process comprising the steps of providing a melt of Ibuprofen and said pharmaceutically acceptable excipient, production of droplets of the melt, and allowing the droplets to cool to form particles, preferably to cool and to come into contact with already solidified particles of the melt to form granules.

According to a particularly preferred embodiment of the present invention the melt granulate according to the present invention is obtainable by a process comprising the following steps
(a) provision of a melt of Ibuprofen and said pharmaceutically acceptable excipient;
(b) production of droplets of the melt by spraying into a processing chamber;
(c) repeated guiding of solid particles past sprayed droplets in the processing chamber with the aid of a process gas jet which is guided in a defined way and whose temperature is fixed, depending on the solidification point of the melt, so that at least some of the droplets come into contact with particles and solidify thereon;
(d) removal of particles from the processing chamber as a function of the particle size.

Said solid particles of step (c) which come into contact with the droplets of the melt are preferably received from a melt having the same composition as the melt the droplets consist of. Preferably, particles of the melt provided according to the process described above and which have already solidified can come into contact with droplets anew.

A melt is produced by melting a substance with heating to a temperature which is typically in the range from 30° C. to 300° C. The melt is preferably obtained by complete melting of a substance or mixture of substances, so that a homogeneous phase is formed. Alternatively, solid substances can be dispersed in the melt. Unless indicated otherwise, the term melt is understood here in this wider sense.

A process gas jet is preferably utilized to guide solid particles repeatedly past sprayed droplets. The process gas may be for example air or an inert gas such as nitrogen, carbon dioxide or a noble gas.

The present invention is also directed to a pharmaceutical composition comprising a melt granulate according to the present invention as defined above, including all preferred embodiments and combinations thereof.

Preferably, the pharmaceutical composition according to the present invention is an oral dosage form.

The granules according to the present invention are suitable for the production of pharmaceutical compositions such as tablets, orally disintegrating tablets, dispersible tablets, effervescent tablets, sachets, stick packs, capsules, inspissated juices and dry suspensions. The preparation of any of these oral dosage forms based on the melt granulate according to the present invention is conventional and is therefore not further described herein. However, the fact that the melt granulate according to the present invention contains Ibuprofen in a taste-masked manner, allows that any further measure of taste-masking is omitted when preparing an oral dosage form. Accordingly, the present invention allows that the melt granulate is filled into sachets or stick packs as it is received from melt granulation. The sachets or stick packs may comprise further components like sweeteners, flavoring agents and/or anti-tacking agents, in an amount of up to 1.0 wt % each, based on the total weight of the oral dosage form, and may also comprise a diluent like mannitol. Similarly, the present invention allows the use of the melt granulate in orally disintegrating tablets, dispersible tablets and effervescent tablets, inspissated juices and dry suspensions. In all these cases formulations with excellent organoleptic properties are achievable based on the taste-masked melt granulate. Finally, in case of conventional tablets there is no need of any taste-masking coating to be applied. This allows the preparation of tablets with higher concentration of Ibuprofen, i.e. for a given dosage of Ibuprofen the tablet size can decrease.

Therefore, preferably, an oral dosage form according to the present invention is selected from the group consisting of tablets, orally disintegrating tablets, dispersible tablets, effervescent tablets, sachets, stick packs, capsules, inspissated juices and dry suspensions.

In case of tablets, orally disintegrating tablets, dispersible tablets, effervescent tablets, sachets, stick packs, capsules and dry suspensions it is preferred according to the present invention that the content of Ibuprofen is at least 50 wt %, more preferably at least 55 wt %, and even more preferably at least 60 wt % based on the total weight of the respective oral dosage form. Such oral dosage forms will usually not comprise more than 90 wt % of Ibuprofen based on the total weight of the respective oral dosage form.

The present invention is also directed to a process for producing taste-masked Ibuprofen granules by hot-melt granulation of Ibuprofen and a pharmaceutically acceptable excipient as defined above.

Preferably, the process comprises the steps of providing a melt of Ibuprofen and said pharmaceutically acceptable excipient, production of droplets of the melt, and allowing the droplets to cool to form particles, preferably to cool and to come into contact with already solidified particles of the melt to form granules.

According to a particularly preferred embodiment of the present invention, the process comprises the following steps:
(a) provision of a melt of Ibuprofen and a pharmaceutically acceptable excipient,
   wherein said pharmaceutically acceptable excipient is selected from the group consisting of fatty acids, fatty acid salts, fatty acid esters, glyceride esters, waxes, polyethylene glycol, and polyvinyl caprolactam-polyvinyl acetate polyethylene glycol graft copolymer,
   wherein said pharmaceutically acceptable excipient is present in the melt in an amount of at least 10 wt % based on the total weight of the melt, and
   wherein Ibuprofen is present in the melt preferably in an amount of at least 60 wt % based on the total weight of the melt;
(b) production of droplets of the melt by spraying into a processing chamber;
(c) repeated guiding of solid particles past sprayed droplets in the processing chamber with the aid of a process gas jet which is guided in a defined way and whose temperature is fixed, depending on the solidification point of the melt, so that at least some of the droplets come into contact with particles and solidify thereon;
(d) removal of particles from the processing chamber as a function of the particle size.

Said solid particles of step (c) which come into contact with the droplets of the melt are preferably received from a melt having the same composition as the melt the droplets consist of. Preferably, particles of the melt provided according to the process described above and which has already solidified can come into contact with droplets anew.

A melt is produced by melting a substance with heating to a temperature which is typically in the range from 30° C. to 300° C. The melt is preferably obtained by complete melting of a substance or mixture of substances, so that a homogeneous phase is formed. Alternatively, solid substances can be dispersed in the melt. Unless indicated otherwise, the term melt is understood here in this wider sense.

A process gas jet is preferably utilized to guide solid particles repeatedly past sprayed droplets. The process gas may be for example air or an inert gas such as nitrogen, carbon dioxide or a noble gas.

The present invention is also directed to the use of a melt granulate as defined above in a pharmaceutical composition, preferably in an oral dosage form.

Finally, the present invention is also directed to the use of a pharmaceutically acceptable excipient as defined above in a melt granulate for providing taste-masked Ibuprofen granules.

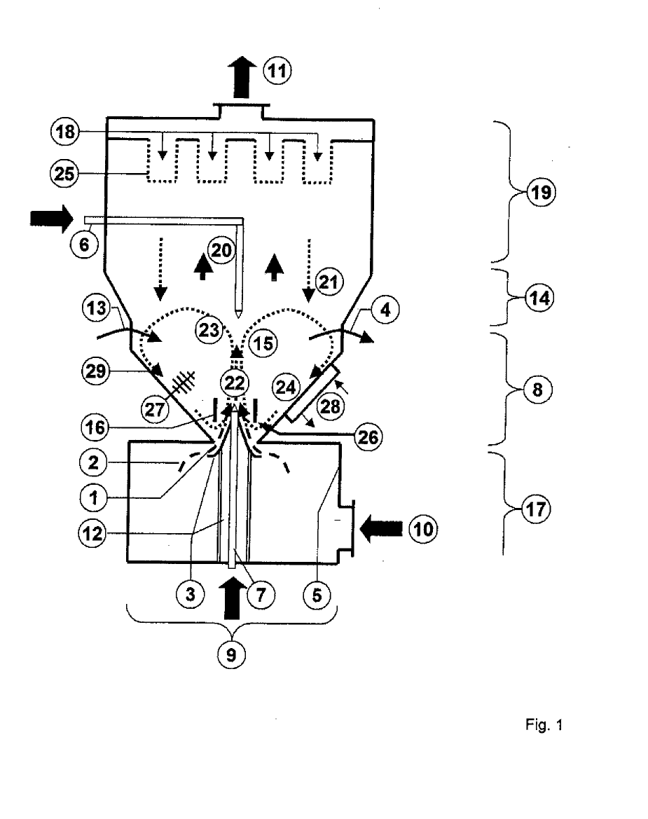

The process for producing taste-masked Ibuprofen granules in its particularly preferred embodiment described above, and the process which is described above in connection with the particularly preferred embodiment of the melt granulate according to the present invention which is obtainable by said process, is preferably carried out in a fluid bed apparatus as described below and shown in FIG. 1. In the examples section it is referred to said apparatus, too. The preferred process and the preferred apparatus are also disclosed in US 2010/0152486 A1.

In the process according to the particular preferred embodiment of the present invention mentioned above, it is made possible to achieve the build-up of globular particles by particles which have been introduced or formed from the melt repeatedly coming into contact with droplets of the melt, so that globular particles of a desired size can be built up. For this purpose, the particles ate moved inside a processing chamber with the aid of a process gas jet guided in a defined way. Particles which have reached a desired size can leave the processing chamber.

The process gas jet is essential both for transport of matter and for transport of heat. Through choice of the temperature of the process gas jet as a function of the solidification point of the melt, it is achieved that contact being made between the sprayed droplets and particles which have already solidified to form substantially globular particles. In particular, the temperature conditions provided in the processing chamber are such as to sufficiently delay solidification in order to make it possible for the particles which are already solid to be wetted with the sprayed droplets of the melt and for globular structures to form. On the other hand, the coming into contact with one another, and adhesion, of particles with a liquid surface is substantially prevented by said particular preferred process.

Accordingly, the process gas jet has a temperature which is below the solidification product of the melt. On the other hand, the temperature of the process gas jet must not permit immediate solidification of droplets sprayed into the processing chamber. The temperature of the process gas jet is preferably 10° C. to 40° C. below the solidification point of the melt.

It is preferred for droplets of the melt and solid particles to be brought into contact with one another in a spouted bed. Spouted bed means that the completely fluidized solid particles are located in a closed solid flow which is stable over time. The spouted bed is generated with the aid of the process gas jet which is guided in a defined way. Three fluidization states or zones are to be distinguished within the spouted bed. In a first zone or ejection zone, the solid particles are accelerated through the action of the process gas jet which is guided in a defined way, and the particles in this zone move in the direction of flow of the process gas jet. Typically, the process gas jet is guided vertically upwards. Correspondingly, the flow prevailing in the ejection zone of the spouted bed is directed vertically upwards. In a subsequent second zone or fountain zone, the particles change their direction of flow. The prevailing flow is transverse. Finally, the particles reach a third zone or return zone. The particles therein then show a motion in the opposite direction until they finally return to the inflow of the gas flow which is guided in a defined way, and are again entrained by the latter in the first zone. The particles move in the return zone typically under the influence of gravity.

The melt can be sprayed through two- or multi-fluid nozzles. A further possibility is to use pressurized nozzles for the spraying. Alternatively, droplet formation is possible by rotary atomizers, jet cutters, ultrasonic droplet formers and other devices known to the skilled person.

It is possible, by spraying droplets of a melt into the processing chamber and allowing these droplets to solidify, to form nuclei of solid particles which are then brought into contact with further droplets in order to form particles of the desired size. An alternative or additional possibility in the method is to supply solid particles from outside. For example, undersized particles which have been removed from the process can be returned as nucleus material to the processing chamber. It is likewise possible for oversized particles which have been removed from the process, or agglomerates of particles, to be comminuted by any desired comminuting unit and returned as nucleus material to the processing chamber. It is also possible to supply particles of different compositions than that of the melt. Melt embedding of the supplied particles is possible in this way.

The particles formed by the particular preferred method of the invention are removed from the processing chamber. The discharge of the finished product material from the processing chamber or a transport of material into a further downstream processing chamber can take place in the region of the transition from the transverse flow to the downwardly directed solid flow. In one embodiment, the particles discharged from the processing chamber are not classified. In another embodiment, the particles discharged from the processing chamber are removed in a classified manner through one or more screening apparatuses.

The method can be carried out for example with the aid of a device as described in DE 103 22 062 A1. The content of the application is incorporated herein by reference.

The method is preferably carried out using a device as shown in the appended FIG. 1. This is explained in detail below.

The amount of process gas 10 (usually heated air) necessary for solidifying the particles to be produced is supplied to an inlet air chamber 17 with rectangular cross section 9 and limiting side walls 5. The process gas 10 is distributed in the inlet air chamber 17 and enters the processing chamber 8 in the form of gas jets 2 through slit apertures 1. The process gas stream which preferably enters the slit 1 horizontally is deflected by the deflecting part 3 preferably upwards into the processing chamber 8 and flows as a type of free jet into the apparatus. Thereafter it is optionally possible for the cross section of the apparatus to become larger in the expansion zone 14 so that the velocity of the process gas flow steadily diminishes upwards. The gas leaves the apparatus as exit gas 11 above the expansion zone 14 over the exit-air part 19 into which it is optionally possible for a dust-removal system (e.g. filter cartridges or textile filter elements) to be integrated.

Present in the processing chamber 8 is an amount of particles which are carried upwards by the process gas jet. In the upper region of the processing chamber 8, and in the expansion zone 14 located above it, the gas velocity decreases, so that the upward-flowing particles leave the gas jet 23 laterally and fall back into the processing chamber 8. The processing chamber 8 is limited in the lower region by inclined side walls 29. Owing to this inclination at the sides, the particles are conveyed under the action of gravity via the return zone 24 in the direction of the gas-inlet slit 1, where they are subsequently carried by the process gas back into the processing chamber 8.

This mechanism results in formation of a very uniform solid circulation 15 consisting of an upward flow and a return in the direction of the process gas inlet. This results in a high particle density in the core zone above the deflecting part 3 even when there are very small amounts of particles in the processing chamber 8. One or more spray nozzles 7 are disposed in this region and spray upwards in the same direction as the process gas jet and serve to introduce the melt.

The high particle loading in the core zone results in very advantageous conditions for heat and material transfer in the nozzle-spraying zone 22. A further consequence is that the melt is very substantially deposited on the particles and thus wets them uniformly on the particle surfaces. The uniform wetting with, at the same time, high solid circulation between nozzle-spraying region and return zone 24 has the effect that a very uniform liquid film is formed. The melt solidifies through the solidification process, and the solid remains on the particle surface. This results in very uniform and homogeneous growth of the granules, leading to a very narrow particle size distribution and a homogeneous particle structure.

The process gas may discharge some of the particles, and fines and dust, as solid-loaded exit air 20 from the processing chamber 8. Deposition of these particles is possible by using the filter system which is optionally integrated in the exit-air part 19, or the dust-removal systems downstream of the apparatus. In the case of an integrated dust-removal system 25, for example, compressed air pulses 18 can be used in order to return the retained particles as removed solid 21 into the processing chamber 8.

Compared with fluidized bed apparatuses with integrated filter systems, the dust recycling is facilitated by the upwards-directed process gas flow being substantially spatially restricted and thus the particles which are to be returned are able reliably to descend outside the gas jet. This mechanism is additionally promoted by the suction effect in the vicinity of the gas-inlet slit 1. Alternatively, particles deposited from the exit air can be returned to the processing chamber 8. For this purpose, various types of feed 26 can be disposed in the lower region of the inclined side walls 29. Owing to the high velocity of the process gas jet in the vicinity of the gas-inlet slit 1, the fine particles are sucked up and supplied to the nozzle-spraying zone 22 where they are wetted with melt and take part in the growth process.

Optionally incorporated guide plates 16 assist the gas jet, enhance the suction effect and improve the feeding of solids into the nozzle-spraying zone 22. Any agglomeration effects which occur are minimized because very high flow velocities and thus greater separation forces than in fluidized beds occur in the nozzle-spraying region. This results in the particles being separated and growing into granules with a globular shape.

The flow profile of the process gas in the processing chamber 8 has the further effect that fine particles returned from the optionally integrated filter system into the processing chamber do not fall back into the nozzle-spraying zone 22. Adhesion of fine particles and consequent agglomeration processes are suppressed thereby.

To carry out the process continuously, the apparatus can optionally be equipped with various input systems 13 for solids. It is possible thereby to supply to the process for example particles which can be obtained by comminuting for example (oversized) granules or/and consist of undersized granules. These particles then serve as granulation nuclei or as initial charge to shorten the operating time. It is additionally possible here for additives which are to be incorporated in the granules to be fed in solid form into the process.

The apparatus can further be provided with discharge elements 4 in order to be able to remove particles from the processing chamber 8. This can take place for example by an overflow or by a volumetric discharge element (e.g. a star wheel discharger) or else by a gravity separator (e.g. a zig-zag classifier or an ascending pipe classifier supplied with screening gas).

It is optionally possible to attach mechanical units 27 in the processing chamber 8, but preferably in the region of the return zone 24 on the inclined walls, in order to generate, by comminution, sufficient fine material as nuclei for the granulation process. The return zone 24 can further optionally be used for siting heating devices or other heat-transfer units 28. For example, the apparatus wall can be jacketed in order to use it for example for heating or cooling the walls by employing liquid or gaseous heat transfer agents. It is thus possible to adjust optimal surface temperatures in order to avoid for example deposits of product.

Spray nozzles 6 which preferably spray downwards, but also partly upwards, can optionally be disposed in the processing chamber 8 or in the parts of the apparatus located above, the expansion zone 14 and the exit-air part 19. The liquid formulation can be sprayed in here likewise in order, for example, to produce granulation nuclei by spray drying/spray congealing in the apparatus. Alternatively, additives or other components in liquid form can be sprayed in through some of the spray units 6 and 7 and thus be incorporated homogeneously into the granular structure. If the spray nozzles 7 pass through the heated inlet air chamber 17, it is optionally possible for the liquid-carrying parts to be provided with insulators or various cooling or heating systems 12 in order to diminish damage to the liquid formulation.

An advantage of the described process is the very simple configuration which combines a high safety of Operation and lack of susceptibility to malfunctioning with very good cleanability. Improved manufacturing conditions, in particular in relation to pharmaceutical and hygiene requirements on change of product, are thus created.

### Measurement Methods and Definitions

Dissolution Performance

Dissolution testing were conducted in accordance with the USP method and according to the dissolution testing guidelines “Dissolution Testing of Immediate Release Solid Oral Dosage Forms” (Guidance for Industry of the U.S. Department of Health and Human Services, Food and Drug Administration, Center for Drug Evaluation and Research, August 1997) using dissolution apparatus type II (paddle) (Sotax, model AT7, Switzerland) in 900 ml of buffered phosphate pH 7.2±0.05 and at 37±0.5° C. temperature. The paddle was driven at 50 rpm rotation speed. The samples for dissolution testing always reflected 200 mg Ibuprofen drug loading. The cumulative drug release was determined online using a UV spectrophotometer Perkin-Elmer Lambda 25 (Perkin-Elmer, USA) operating at 221 nm, after filtration through a glass microfiber filter Whatman GF/D (Whatman, UK). Samples were withdrawn over a 720 minutes period at predetermined time: 3, 7, 11, 15, 19, 24, 30, 45, 60, 75, 90, 105 and 120 minutes respectively.

Samples (melt granulates or oral dosage forms) according to the present invention qualify as having immediate release properties if not less than 80% of the Ibuprofen load is dissolved in 60 minutes.

Assay/Purity

Instrumentation

The experiments were carried out using an Agilent 1100/1200 (Agilent, USA) equipped with a quaternary gradient pump, a diode array detector, an autosampler with Rheodyne injection valve and a thermostated column. The Agilent 1100/1200 was fully controlled by the Waters Empower 2 Software.

Conditions

Ibuprofen samples were analyzed using a Luna C 18(2) column (5 μm, length 150 mm, internal diameter 3.0 mm) (Phenomenex, Switzerland) and a C 18 pre-column 4.0 mm×3.0 mm. The mobile phase used for the gradient method was composed of a mixture of acetonitrile (mobile phase A) and phosphate buffer solution pH 2.5±0.05 (mobile phase B) with 0.7 ml/min flow rate (details on the gradient method exposed in the table below) and 30° C. column temperature (20° C. auto sampler temperature). The Ibuprofen content was determined at a wavelength of 254 nm with an injection volume of 5 μL. The impurities were determined at a wavelength of 214 nm with an injection volume of 1 μL. The chromatogram time was 20 minutes.

| Time/min | Mobile phase A/% | Mobile phase B/% |
|---|---|---|
| 0.0 | 38 | 62 |
| 9.0 | 58 | 42 |
| 15.0 | 58 | 42 |
| 15.1 | 38 | 62 |
| 20.0 | 38 | 62 |

Particle Size Distribution

The particle size distribution of the pellets was determined by sieve analysis performed with a Retsch AS 200 control ‘g’ (Retsch, Germany) operating at the following conditions: 50 g sample size, 1.5 amplitude and 10 minutes sieving time.

Bulk and tapped Densities

Bulk Density

The bulk density was determined using an ERWEKA Tapped Density Tester (Erweka SMV20). Approximately 100 g (m) of the test sample were introduced into a dry graduated cylinder of 250 ml without compacting. The unsettled apparent volume (V0) to the nearest graduated unit was read and the bulk density in g/ml was calculated by the formula m/V0.

Tapped Density

The tapped density was determined using an ERWEKA Tapped Density Tester (Erweka SMV20). 10, 500 and 1250 taps were carried out on the same powder sample used for the bulk density determination and the corresponding volumes V10, V500 and V1250 to the nearest graduated unit were read. If the difference between V500 and V1250 was less than or equal to 2 ml, V1250 was considered as the tapped volume. If the difference between V500 and V1250 exceeded 2 ml, such as 1250 taps were repeated in increments, until the difference between succeeding measurements was less than or equal to 2 ml. The tapped density in g/ml was calculated using the formula m/Vf in which Vf is the final tapped volume.

### EXAMPLES

In the following examples the preparation of granules according to the present invention is demonstrated. In all examples a fluid bed apparatus as described above and shown in FIG. 1 is used. Its processing chamber has a rectangular cross section and has a cross-sectional area of 0.15×0.2=0.03 m² and a height of 1 m above the inclined side walls. The process air stream is supplied through two gas distribution cylinders running lengthwise through the apparatus. The melt is sprayed in through a binary nozzle spraying vertically upwards and fed with compressed air into the processing chamber with a certain mass flow rate. The diameter of the spray nozzle is 1.2 mm. Dust is removed from the outlet air of the apparatus by a cyclone separator, and the deposited solid is fed gravimetrically into the process chamber in the vicinity of the slit as nucleus material. A zig-zag classifier is used to remove granules continuously from the front of the processing chamber. The fines separated in the classifier are blown back by the screening air into the processing chamber.

The process parameters of the following examples are summarized in tables 1 and 2.

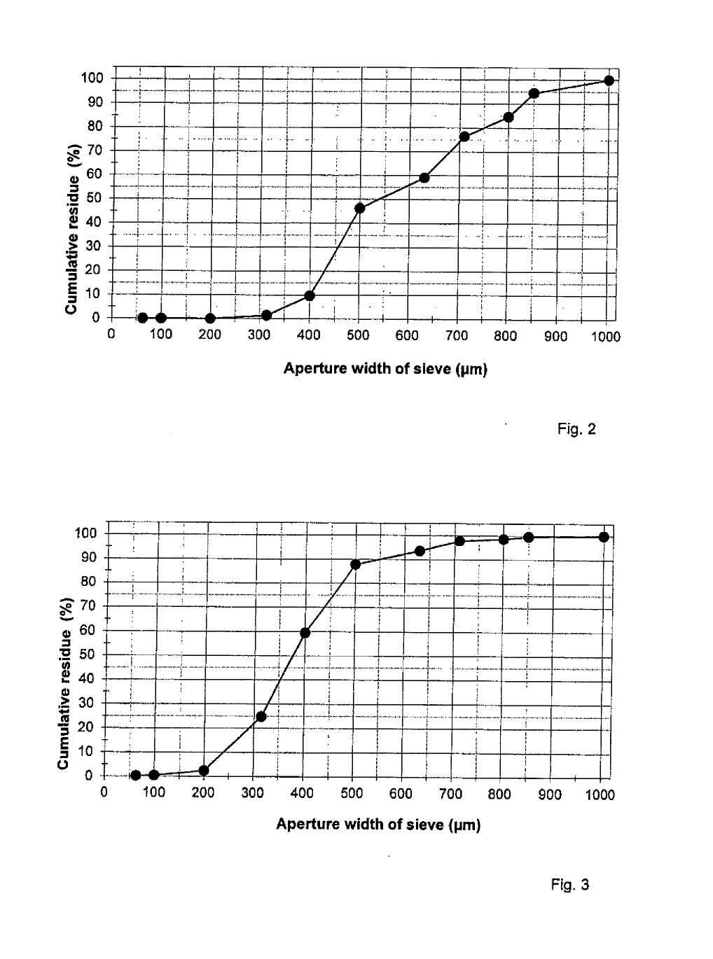

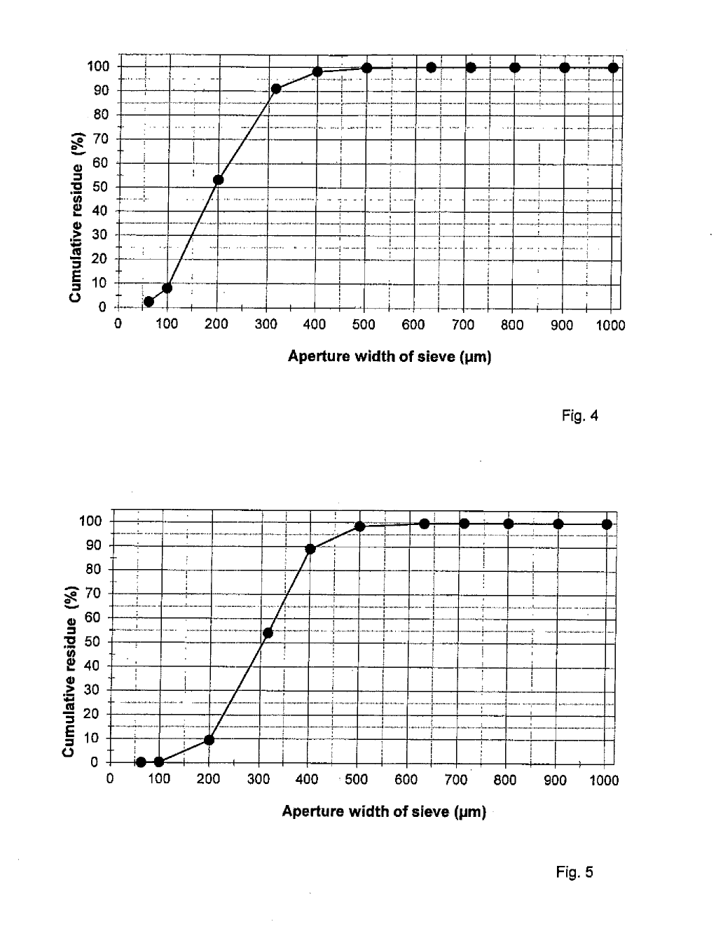

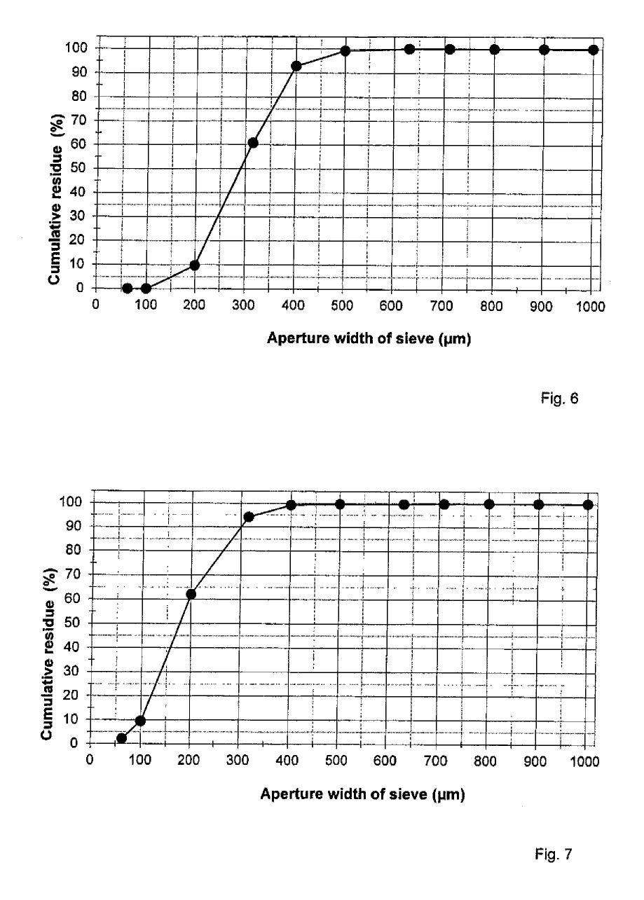

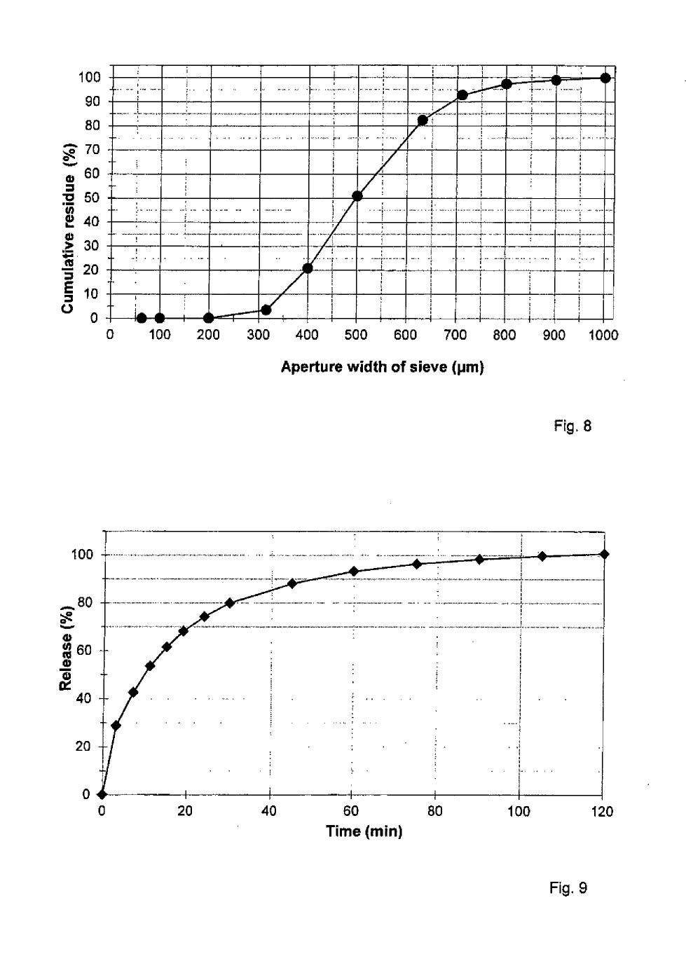

The granules received from the classifier according to the present examples have a tapped density and a main particle size fraction (i.e. particle size fraction containing at least 90 wt % of the granules) as shown below in tables 3 and 4. The particle size distribution of the granules measured by sieve analysis is shown in FIGS. 2 to 8. Hence, the granules have a uniform particle size distribution. The fractions received from sieve analysis are shown in table 5.

The measured Ibuprofen content of the granules is given in tables 3 and 4 and compared with the theoretical content. The relative standard deviation is also given.

The granules have a length/width ratio of less than 1.4, they are very hard, and they have a relatively smooth and glossy surface.

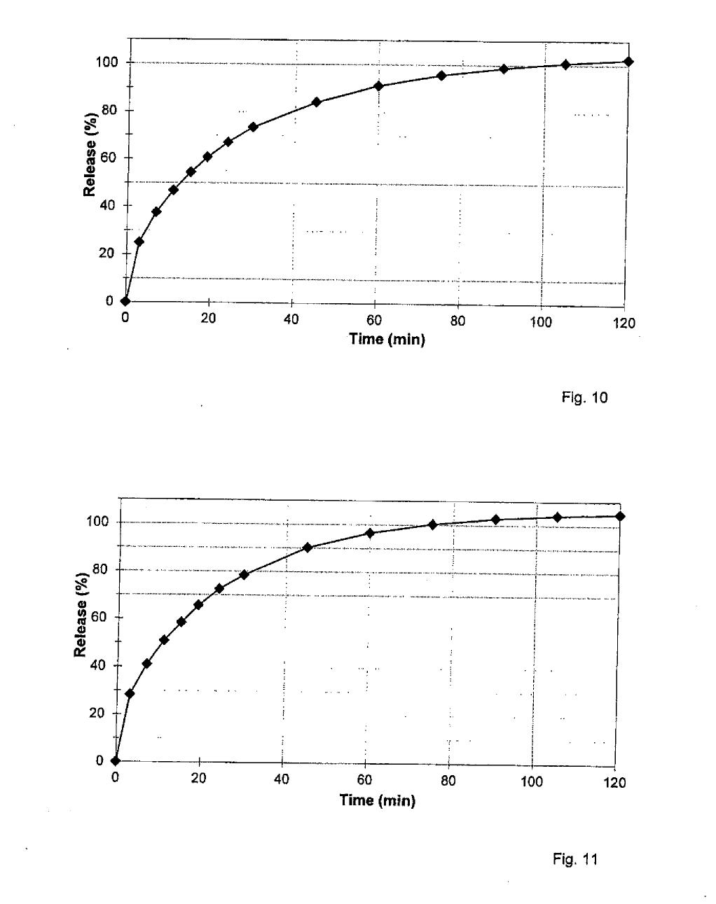

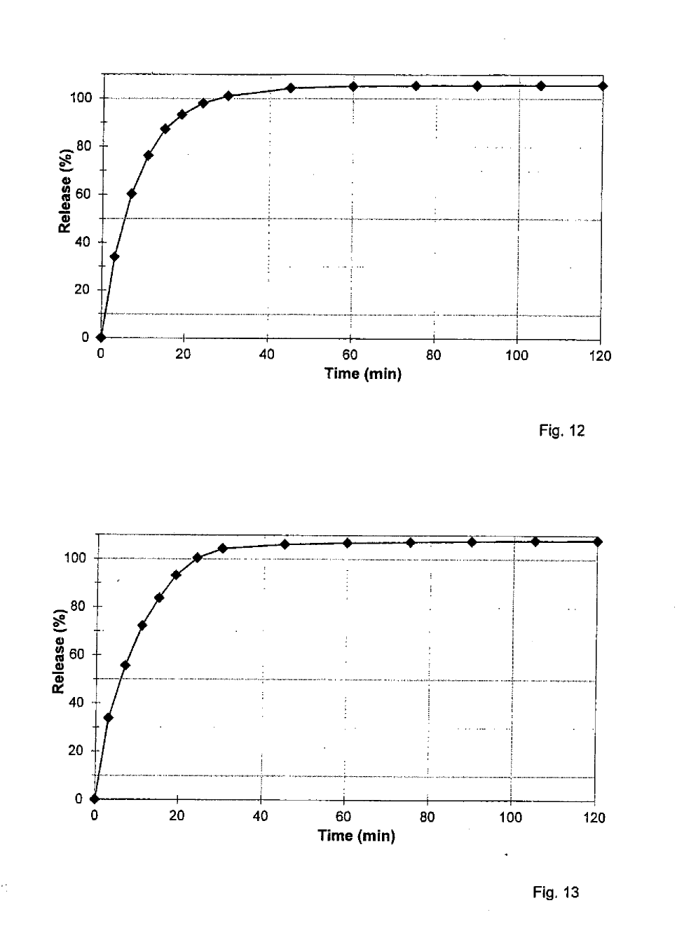

The cumulative release of the granules is shown in FIGS. 9 to 13. The profiles show immediate release of Ibuprofen according to the requirements of the USP (U.S. Pharmacopeia) for immediate release Ibuprofen products.

Values of purity of the granules ate shown in table 6.

### Example 1

The pharmaceutical excipient stearic acid (about 2.5 kg) is placed in a container made of stainless steel and melted at a temperature of 80° C. About 7.5 kg of the active pharmaceutical ingredient Ibuprofen is added to the melt and melted therein, wherein the amount of Ibuprofen is such that its fraction is 75 wt % of the final melt. The mixture is heated and stirred continuously during the whole granulation process. The melt is then sprayed into the processing chamber of an apparatus as described above. The mass flow rate of the melt is approximately 20-25 g/min. The temperature of the spraying air is 90° C. The process air stream is supplied at a temperature of 25° C. and at 130 m³/h.

### Example 2

The pharmaceutical excipient stearic acid (about 1.0 kg) is placed in a container made of stainless steel and melted at a temperature of 80° C. About 9.0 kg of the active pharmaceutical ingredient Ibuprofen is added to the melt and melted therein, wherein the amount of Ibuprofen is such that its fraction is 90 wt % of the final melt.

The further process is carried out as described for Example 1, but with the process parameters as shown in table 1.

### Example 3

The pharmaceutical excipient Carnauba wax (about 2.5 kg) is placed in a container made of stainless steel and melted at a temperature of 110° C. About 7.5 kg of the active pharmaceutical ingredient Ibuprofen is added to the melt and melted therein, wherein the amount of Ibuprofen is such that its fraction is 75 wt % of the final melt.

The further process is carried out as described for Example 1, but with the process parameters as shown in table 1.

### Example 4

The pharmaceutical excipient Carnauba wax (about 2.0 kg) is placed in a container made of stainless steel and melted at a temperature of 110° C. About 8.0 kg of the active pharmaceutical ingredient Ibuprofen is added to the melt and melted therein, wherein the amount of Ibuprofen is such that its fraction is 80 wt % of the final melt.

The further process is carried out as described for Example 1, but with the process parameters as shown in table 1.

### Example 5

The pharmaceutical excipient Carnauba wax (about 1.5 kg) is placed in a container made of stainless steel and melted at a temperature of 110° C. About 8.5 kg of the active pharmaceutical ingredient Ibuprofen is added to the melt and melted therein, wherein the amount of Ibuprofen is such that its fraction is 85 wt % of the final melt.

The further process is carried out as described for Example 1, but with the process parameters as shown in table 2.

### Example 6

The pharmaceutical excipient Carnauba wax (about 1.0 kg) is placed in a container made of stainless steel and melted at a temperature of 110° C. About 9.0 kg of the active pharmaceutical ingredient Ibuprofen is added to the melt and melted therein, wherein the amount of Ibuprofen is such that its fraction is 90 wt % of the final melt.

The further process is carried out as described for Example 1, but with the process parameters as shown in table 2.

### Example 7

The active pharmaceutical ingredient Ibuprofen (about 8.0 kg) is placed in a container made of stainless steel and melted at a temperature of 110° C. About 2.0 kg of the pharmaceutical excipient Soluplus® is added to the melt and melted therein, wherein the amount of Soluplus® is such that the fraction of Ibuprofen is 80 wt % of the final melt.

The further process is carried out as described for Example 1, but with the process parameters as shown in table 2.

Soluplus® is commercially available by BASF and is a polyvinyl caprolactam-polyvinyl acetate-polyethylene glycol graft copolymer. The CAS-No. is 402932-23-4.

The granules of all examples have appropriate taste properties, i.e. they contain Ibuprofen in a fully taste-masked manner.

While this invention has been described in conjunction with the specific embodiments outlined above, it is evident that many alternatives, modifications, and variations will be apparent to those skilled in the art. Accordingly, the preferred embodiments of the invention as set forth above are intended to be illustrative, not limiting. Various changes may be made without departing from the spirit and scope of the inventions as defined in the following claims.

TABLE 1

| Example | | 1 | 2 | 3 | 4 |
|---|---|---|---|---|---|
| Process air volume | m³/h | 130 | 120 | 120 | 120 |
| Process air temperature | ° C. | 25 | 15 | 25 | 25 |
| Outlet air temperature | ° C. | 28 | 25 | 30 | 25 |
| Product temperature | ° C. | 28 | 20 | 30 | 27 |
| Atomizing air pressure | bar | 2.0 | 3.5 | 3.0 | 2.0 |
| Spray mass flow rate | g/min | 20-25 | 20-25 | 30-35 | 20-25 |
| Atomizing air temperature | ° C. | 90 | 90 | 90 | 90 |

TABLE 2

| Example | | 5 | 6 | 7 |
|---|---|---|---|---|
| Process air volume | m³/h | 120 | 130 | 120 |
| Process air temperature | ° C. | 25 | 25 | 23 |
| Outlet air temperature | ° C. | 25 | 27 | 25 |
| Product temperature | ° C. | 30 | 27 | 28 |
| Atomizing air pressure | bar | 2.0 | 3.5 | 4.0 |
| Spray mass flow rate | g/min | 20-25 | 25 | 15-20 |
| Atomizing air temperature | ° C. | 90 | 90 | 115 |

TABLE 3

| Example | | 1 | 2 | 3 | 4 |
|---|---|---|---|---|---|
| Tapped density | g/ml | 0.58 | 0.59 | 0.55 | 0.56 |
| Main particle size fract. | μm | 300-900 | 200-800 | 100-400 | 100-450 |
| Ibuprofen content | | | | | |
| measured | wt % | 78.77 | — | 76.84 | 80.00 |
| rel. to aimed value | % | 105.03 | — | 102.46 | 100.00 |
| Rel. Standard Deviation | % | 0.62 | — | 0.73 | 0.24 |

TABLE 4

| Example | | 5 | 6 | 7 |
|---|---|---|---|---|
| Tapped density | g/ml | 0.51 | 0.53 | 0.32 |
| Main particle size fract. | μm | 100-450 | 100-400 | 200-800 |
| Ibuprofen content | | | | |
| measured | wt % | 85.32 | — | 84.56 |
| rel. to aimed value | % | 100.38 | — | 105.70 |
| Rel. Standard Deviation | % | 0.19 | — | 0.71 |

TABLE 5

| Aperture | Example | | | | | | |
|---|---|---|---|---|---|---|---|
| width of | 1 | 2 | 3 | 4 | 5 | 6 | 7 |
| sieve/μm | Cumulative residue/wt % | | | | | | |
| 63 | 0.00 | 0.20 | 2.44 | 0.10 | 0.00 | 2.16 | 0.01 |
| 100 | 0.00 | 0.40 | 8.04 | 0.30 | 0.00 | 9.43 | 0.03 |
| 200 | 0.00 | 2.40 | 53.20 | 9.40 | 9.70 | 62.04 | 0.14 |
| 315 | 1.40 | 24.80 | 91.08 | 54.00 | 60.80 | 94.14 | 3.52 |
| 400 | 9.60 | 59.50 | 98.19 | 88.90 | 92.90 | 99.19 | 20.87 |
| 500 | 46.20 | 87.80 | 99.60 | 98.40 | 99.20 | 99.64 | 50.87 |
| 630 | 59.00 | 93.60 | 99.87 | 99.60 | 100.00 | 99.69 | 82.31 |
| 710 | 76.40 | 97.80 | 99.87 | 99.80 | 100.00 | 99.82 | 92.78 |
| 800 | 84.60 | 98.60 | 100.00 | 99.80 | 100.00 | 100.00 | 97.26 |
| 850 | 94.40 | 99.60 | n.d. | n.d. | n.d. | n.d. | n.d. |
| 900 | n.d. | n.d. | 100.00 | 99.80 | 100.00 | 100.00 | 98.88 |
| 1000 | 100.00 | 100.00 | 100.00 | 99.80 | 100.00 | 100.00 | 100.00 |

n.d.: not determined

TABLE 6

| | Example | | | | | |
|---|---|---|---|---|---|---|
| Retention | 1 | 3 | 4 | 5 | 6 | 7 |
| time/min | Impurity content/% | | | | | |
| 0.43 | 0.06 | n.d. | n.d. | n.d. | <LOQ | <LOQ |
| 0.62 | 0.06 | 0.06 | n.d. | 0.06 | <LOQ | n.d. |
| 0.63 | n.d. | n.d. | n.d. | n.d. | n.d. | 0.06 |
| 1.29 | 0.07 | 0.06 | n.d. | n.d. | 0.07 | n.d. |

LOQ: Peak area below the limit of quantification
n.d.: No peak detected

### REFERENCE NUMBERS

1 Slit aperture(s)
2 Gas jet(s)
3 Deflecting part
4 Discharge element
5 Side wall
6 Spray nozzle(s) spraying in any directions
7 Spray nozzle(s) spraying upwards
8 Processing chamber
9 Cross section of a process stage
10 Process gas
11 Exit gas
12 Insulator with cooling or heating system
13 Input system
14 Expansion zone
15 Solid circulation
16 Guide plate(s)
17 Inlet air chamber
18 Pulses of compressed air
19 Exit air part
20 Solid-loaded exit air
21 Removed and returned solid
22 Nozzle-spraying zone
23 Particle exit from the gas jet
24 Return zone
25 Dust removal system
26 Feeds
27 Mechanical comminuting units
28 Heat-transfer units
29 Side wall

1. A melt granulate comprising:
Ibuprofen; and
a pharmaceutically acceptable excipient;
wherein said pharmaceutically acceptable excipient is selected from the group consisting of:
fatty acids, fatty acid salts, fatty acid esters, glyceride esters, waxes, polyvinyl caprolactam-polyvinyl acetate polyethylene glycol graft copolymer, and a combination of two or more thereof;
wherein said pharmaceutically acceptable excipient is present in the melt granulate in an amount of at least 10 wt % based on the total weight of the melt granulate; and
wherein Ibuprofen is present in the melt granulate in an amount of at least 60 wt % based on the total weight of the melt granulate.

2. The melt granulate according to claim 1;
wherein the pharmaceutically acceptable excipient is present in the melt granulate in an amount of at least 12 wt % based on the total weight of the melt granulate.

3. The melt granulate according to claim 1;
wherein Ibuprofen is present in the melt granulate in an amount of at least 65 wt % based on the total weight of the melt granulate.

4. The melt granulate according to claim 1;
wherein the sum of the amount of Ibuprofen and the amount of said pharmaceutically acceptable excipient is at least 90 wt % based on the total weight of the melt granulate.

5. The melt granulate according to claim 1;
wherein the pharmaceutically acceptable excipient is selected from the group consisting of
saturated fatty acids containing between 8 and 26 carbon atoms,
fatty acid salts of saturated fatty acids which contain between 8 and 26 carbon atoms,
fatty acid esters of saturated fatty acids which contain between 8 and 26 carbon atoms,
natural glyceride esters of saturated fatty acids which contain between 8 and 26 carbon atoms each,
plant waxes which comprise, in an amount of at least 80 wt % based on the total weight of the wax, one or more compounds selected from the group consisting of the following four classes of compounds:
aliphatic esters, diesters of 4 hydroxycinnamic acid, Ω-hydroxycarboxylic acids, and fatty acid alcohols;
where the alcohol and acid parts in the members of said four classes of compounds contain between 26 and 38 carbon atoms each,
polyvinyl caprolactam-polyvinyl acetate polyethylene glycol graft copolymer; and
a combination of two or more thereof.

6. The melt granulate according to claim 1;
wherein the pharmaceutically acceptable excipient has a melting point of between 40 and 150° C.

7. The melt granulate according to claim 1;
wherein the melt granulate comprises less than 10 wt % based on the total weight of the melt granulate of any hydrophilic compound.

8. The melt granulate according to claim 1;
wherein the melt granulate comprises at least 90 wt % based on the total weight of the melt granulate of hydrophobic compounds or hydrophobic and amphiphilic compounds.

9. A method of obtaining the melt granulate according to any claim 1, comprising the following steps
(a) providing a melt of Ibuprofen and said pharmaceutically acceptable excipient;
(b) producing droplets of the melt by spraying the melt into a processing chamber;
(c) repeatedly guiding solid particles past the sprayed droplets of the melt in the processing chamber with the aid of a process gas jet which is guided in a defined way and whose temperature is fixed, depending on the solidification point of the melt, so that at least some of the droplets come into contact with particles and solidify thereon;
(d) removing particles from the processing chamber as a function of the particle size.

10. A pharmaceutical composition comprising:
the melt granulate according to claim 1.

11. The pharmaceutical composition according to claim 10;
wherein the pharmaceutical composition is an oral dosage form.

12. The pharmaceutical composition according to claim 11;
wherein the oral dosage form is selected from the group consisting of:
tablets, orally disintegrating tablets, dispersible tablets, effervescent tablets, sachets, stick packs, capsules, inspissated juices, and dry suspensions.

13. The pharmaceutical composition according to claim 12;
wherein the oral dosage form is selected from the group consisting of:
tablets, orally disintegrating tablets, dispersible tablets, effervescent tablets, sachets, stick packs, capsules, and dry suspensions; and
wherein the content of Ibuprofen is at least 50 wt % based on the total weight of the respective oral dosage form.

14. A process for producing taste-masked Ibuprofen granules, comprising the following steps:
(a) providing a melt of Ibuprofen and a pharmaceutically acceptable excipient, wherein:
said pharmaceutically acceptable excipient is selected from the group consisting of fatty acids, fatty acid salts, fatty acid esters, glyceride esters, waxes, polyethylene glycol, and polyvinyl caprolactam-polyvinyl acetate polyethylene glycol graft copolymer; and
said pharmaceutically acceptable excipient is present in the melt in an amount of at least 10 wt % based on the total weight of the melt;
(b) producing droplets of the melt by spraying the melt into a processing chamber;
(c) repeatedly guiding solid particles past the sprayed droplets of the melt in the processing chamber with the aid of a process gas jet which is guided in a defined way and whose temperature is fixed, depending on the solidification point of the melt, so that at least some of the droplets come into contact with particles and solidify thereon; and
(d) removing particles from the processing chamber as a function of the particle size.

15. A method comprising:
utilizing the melt granulate according to claim 1 in an oral dosage form.
